(19) United States
(12) Patent Application Publication
(10) Pub. No.: US 2021/0330544 A1
(43) Pub. Date: Oct. 28, 2021

(54) EXTERNAL COUNTERPULSATION SYSTEM AND METHOD OF CONTROLLING SAME

(71) Applicant: OSTEOSYS CO., LTD., Seoul (KR)

(72) Inventors: Young Bok AHN, Seoul (KR); Won Hee LEE, Anyang-si Gyeonggi-do (KR); Jae Jin KIM, Seoul (KR); Jung Tae KIM, Seoul (KR)

(73) Assignee: OSTEOSYS CO., LTD., Seoul (KR)

(21) Appl. No.: 16/622,219

(22) PCT Filed: Jul. 10, 2018
(86) PCT No.: PCT/KR2018/007776

§ 371 Date: Dec. 12, 2019

(30) Foreign Application Priority Data
Jul. 9, 2018 (KR) 10-2018-0079261

(51) Int. Cl. A61H 9/00

(52) U.S. Cl. CPC A61H 9/0007; A61H 9/0092; A61H 2230/255; A61H 2201/1238; A61H 2230/045; A61H 2201/1207

(57) ABSTRACT

The present invention relates to an external counterpulsation system that senses biosignals to monitor a heart condition and performs external counterpulsation on the basis of the monitored heart condition. The external counterpulsation system includes: a compression device wrapping around limbs of a subject to compress or decompress the wrapped limbs; and a controller configured to calculate compression timing and decompression timing for the external counterpulsation by analyzing an electrocardiogram signal and a blood flow rate signal among the sensed biosignals, and control the compression device on the basis of the calculated compression timing and decompression timing. As described above, the present invention can automatically control compression timing and decompression timing, thus ensuring that, unlike the related art technology, the problem of inconvenience that an operator has to control the compression and decompression timings while continuously monitoring procedure is solved, and that operators without specialized knowledge can use the system easily.

### TECHNICAL FIELD

The present invention relates to an external counterpulsation system that repeatedly compresses or decompresses limbs on the basis of an electrocardiogram, thus improving cardiovascular disease, and to a method of controlling the same.

### BACKGROUND ART

An external counterpulsation system is used to perform non-invasive, safe, low-cost, and high efficiency treatment for ischemic heart disease. The external counterpulsation system is a system that treats symptoms such as angina, myocardial infarction, and the like without surgical intervention by increasing the blood flow of dormant blood vessels. The external counterpulsation system compresses or decompresses limbs according to cardiac cycle.

The external counterpulsation system involves the use of external compressive cuffs placed around a patient's lower limbs. While the patient lies on a table with the cuffs placed around his or her calves, thighs, and buttocks, the cuffs inflate during diastole and deflate right before systole. These inflations and deflations repeat regularly in synchronization with a patient's cardiac cycle.

One example of the external counterpulsation system may include a controller, a pneumatic compressor, a set of solenoid valves, and an inflation device. The controller performs treatment by controlling inflation/deflation of the inflation device through control of the pneumatic compressor and the set of solenoid valves according to a sensed cardiac cycle.

In other words, a controller of the external counterpulsation system generates a timing control signal for controlling a compressive cuff to compress and decompress the limbs according to a cardiac cycle of a subject after initial compression intensity is set initially by an operator. Herein, the timing control signal is a very important variable for improvement of symptoms of ischemic heart disease expected through the use of the external counterpulsation system.

In this regard, the external counterpulsation system is configured to sense biosignals of the subject according to compression and decompression procedure operations to display the biosignals to the operator or the like, and to allow the operator to view the displayed biosignals and control the timing control signal for compression and decompression.

However, the external counterpulsation system in the related art as above has a limitation in use because the operator has to be present during a procedure and has to have specialized knowledge to interpret the displayed biosignals in order to control compression timing. In addition, there is a risk that an error may occur during manual control by the operator.

Moreover, the external counterpulsation system in the related art is impossible to change the compression intensity during a procedure when once the compression intensity is set initially. This leads to reduced therapeutic effect and causes pain in the subject in some cases.

### DISCLOSURE

### Technical Problem

Accordingly, the present invention has been made keeping in mind the above problems occurring in the related art, and an objective of the present invention is to provide an external counterpulsation system that automatically controls timing of compression and decompression of limbs or compression intensity during a procedure and reduces noise and vibration problems occurring in a conventional pneumatic drive method, and to provide a method of controlling the same.

### Technical Solution

In order to accomplish the above objective, according to one aspect of the present invention, there is provided an external counterpulsation system that senses biosignals to monitor a heart condition and performs external counterpulsation on the basis of the monitored heart condition.

The external counterpulsation system may include: a compression device wrapping around limbs of a subject to compress or decompress the wrapped limbs; and a controller configured to calculate compression timing and decompression timing for the external counterpulsation by analyzing an electrocardiogram signal and a blood flow rate signal among the sensed biosignals, and control the compression device on the basis of the calculated compression timing and decompression timing.

The controller may calculate a blood flow rate reference waveform for a blood flow rate during late diastole and systole, the blood flow rate being achievable by performing the external counterpulsation, using a result of analyzing the electrocardiogram signal and the blood flow rate signal, control the compression timing and the decompression timing for the external counterpulsation system such that a waveform of the blood flow rate signal of the sensed biosignals matches the blood flow rate reference waveform, and control the compression device on the basis of the controlled compression timing and decompression timing.

The controller may configure a peak value of a blood flow rate signal during late diastole in the blood flow rate reference waveform as a first reference value, configure a valley value of the blood flow rate signal during systole in the blood flow rate reference waveform as a second reference value, and control the compression timing and the decompression timing such that a value during late diastole in the waveform of the blood flow rate signal of the sensed biosignals matches the first reference value and a value during systole in the waveform of the blood flow rate signal of the sensed biosignals matches the second reference value.

The controller may divide an amplitude of the blood flow rate signal into a level L1, a level L2, and a level L3 in order from the highest to the lowest, control the compression timing by retarding the compression timing when a value of the blood flow rate signal at the beginning of late diastole is between the level L1 and the level L2 and a value of the blood flow rate signal at the end of late diastole is near the first reference value, and control the compression timing by advancing the compression timing when the value of the blood flow rate signal at the beginning of late diastole is between the level L2 and the level L3 and the value of the blood flow rate signal at the end of late diastole is less than the first reference value.

The controller may control the decompression timing by retarding the decompression timing when a value of the blood flow rate signal at the beginning of systole and a value of the blood flow rate signal at the end of systole are near the level L3, and control the decompression timing by advancing the decompression timing when the value of the blood flow rate signal at the beginning of systole is near the level L2 and the value of the blood flow rate signal at the end of systole is between the level L2 and the level L3.

The controller may configure compression intensity on the basis of a preconfigured initial maximum compression value and start performing the external counterpulsation on the basis of the configured compression intensity, and the controller may control a value of the configured compression intensity by analyzing the electrocardiogram signal and the blood flow rate signal of the sensed biosignals and control the compression device to perform the external counterpulsation on the basis of the controlled compression intensity.

The controller may calculate a peak value of the blood flow rate signal at the end of late diastole of the subject using a result of analyzing the electrocardiogram signal and the blood flow rate signal, control the compression intensity by decreasing the compression intensity when the calculated peak value of the blood flow rate signal is greater than a predetermined value, and control the compression intensity by increasing the compression intensity with the initial maximum compression value as an upper limit when the calculated peak value of the blood flow rate signal is less than the predetermined value.

The initial maximum compression value may be configured by controlling the compression device to apply the test compression to the limbs for the predetermined test period with the predetermined test intensity before performing the external counterpulsation.

According to another aspect of the present invention, there is provided a method of controlling an external counterpulsation system that senses biosignals to monitor a heart condition and performs external counterpulsation on the basis of the monitored heart condition by controlling a compression device wrapping around limbs of a subject to compress and decompress the wrapped limbs.

The method may include: analyzing an electrocardiogram signal and a blood flow rate signal among the sensed biosignals; calculating a blood flow rate reference waveform for a blood flow rate during late diastole and systole, the blood flow rate being achievable by performing the external counterpulsation, using a result of analyzing the electrocardiogram signal and the blood flow rate signal; controlling compression timing time and decompression timing for the external counterpulsation; and controlling the compression device on the basis of the controlled compression timing and decompression timing.

The method may further include: configuring compression intensity on the basis of a preconfigured initial maximum compression value and starting performing the external counterpulsation on the basis of the configured compression intensity; and controlling the configured compression intensity by using a result of analyzing the electrocardiogram signal and the blood flow rate signal and performing the external counterpulsation on the basis of the controlled compression intensity.

### Advantageous Effects

As described above, the present invention can automatically control compression timing and decompression timing, thus ensuring that, unlike the related art technology, the problem of inconvenience that an operator has to control the compression and decompression timings while continuously monitoring procedure is solved, and that operators without specialized knowledge can use the system easily.

Furthermore, the present invention can automatically configure an initial maximum compression value through test compression, thus ensuring that, unlike the related art technology, the presence of the operator is not necessarily required and an error that may occur when the initial compression intensity is manually input is avoided. The present invention also can configure the initial compression intensity at maximum level to be personalized to a subject, thus ensuring that enhanced therapeutic effect of external counterpulsation is provided.

Furthermore, the present invention can automatically control compression intensity applied to the limbs during a procedure. This ensures that, unlike the related art technology, the problem of pain in the subject caused by inability to automatically control the compression intensity during a procedure is solved, and that enhanced therapeutic effect is provided.

Furthermore, the present invention can implement a compression device for compressing and decompressing the limbs by an electric drive method, thus ensuring that noise and vibration are reduced compared to a conventional hydraulic drive method.

### DESCRIPTION OF DRAWINGS

FIG. 1 is a block diagram showing an external counterpulsation system according to an embodiment of the present invention.

FIG. 2 is a block diagram showing a biosignal sensing device according to an embodiment of the present invention.

FIG. 3 is a block diagram showing a compression device according to an embodiment of the present invention.

FIG. 4 is a block diagram showing a blood flow rate reference waveform according to an embodiment of the present invention.

FIGS. 5 and 6 are views showing control of compression timing according to an embodiment of the present invention.

FIGS. 7 and 8 are views showing control of decompression timing according to an embodiment of the present invention.

FIG. 9 is a view showing compression and decompression actions of a controller according to an embodiment of the present invention.

FIG. 10 is a flowchart showing a method of controlling an external counterpulsation system according to an embodiment of the present invention.

FIG. 11 is a flowchart showing a step of controlling compression and decompression timing and compression intensity according to an embodiment of the present invention.

### BEST MODE

The above and other objectives, features, and advantages of the present invention will be more clearly understood from the following detailed description taken in conjunction with the accompanying drawings, such that the invention can be easily embodied by one of ordinary skill in the art to which this invention belongs. In the following description, it is to be noted that, when the functions of conventional elements and the detailed description of elements related with the present invention may make the gist of the present invention unclear, a detailed description of those elements will be omitted.

It will be understood that, although the terms first, second, etc. may be used herein to describe various elements, these elements should not be limited by these terms. These terms are only used to distinguish one element, from another element. For instance, a first element discussed below could be termed a second element without departing from the teachings of the present invention. Similarly, the second element could also be termed the first element. The terminology used herein is for the purpose of describing particular embodiments only and is not intended to be limiting of example embodiments of the invention. As used herein, the singular forms “a”, “an”, and “the” are intended to include the plural forms as well, unless the context clearly indicates otherwise.

Although terminologies used in the present specification are selected from general terminologies used currently and widely in consideration of functions, they may be changed in accordance with intentions of technicians engaged in the corresponding fields, customs, advents of new technologies, and the like. Occasionally, some terminologies may be arbitrarily selected by the applicant(s). In this case, the meanings of the arbitrarily selected terminologies shall be described in the corresponding part of the detailed description of the specification. Therefore, terminologies used in the present specification need to be construed on the basis of the substantial meanings of the corresponding terminologies and the overall matters disclosed in the present specification rather than construed as simple names of the terminologies.

Unless the context clearly indicates otherwise, it will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Also, the terms “˜ part”, “˜ unit”, “module”, “apparatus”, and the like mean a unit for processing at least one function or operation and may be implemented by a combination of hardware and/or software.

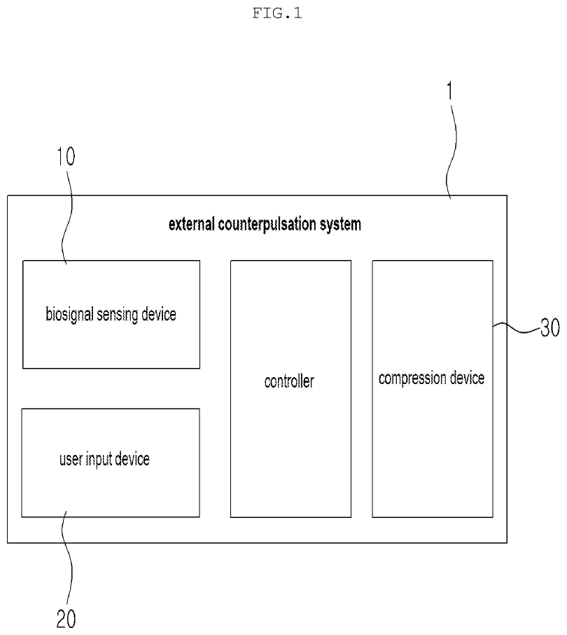

Hereinafter, an external counterpulsation system according to an embodiment of the present invention will be described with reference to FIGS. 1 to 4.

As shown in FIG. 1, an external counterpulsation system 1 according to the present embodiment includes a biosignal sensing device 10, a user input device 20, a compression device 30, and a controller 40 in order to sense biosignals to monitor a heart condition, and perform external counterpulsation on the basis of the monitored heart condition.

The biosignal sensing device 10 is a device for measuring biosignals of a subject undergoing treatment related to external counterpulsation treatment. The biosignals measured as above are transmitted to the controller 40.

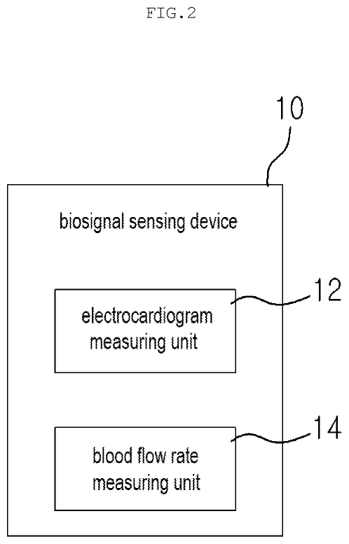

Hereinafter, the biosignal sensing device 10 will be described in detail with reference to FIG. 2.

As shown in FIG. 2, the biosignal sensing device 10 is a device for measuring biosignals of a subject undergoing treatment related to external counterpulsation treatment. The biosignal sensing device may include an electrocardiogram measuring unit 12 and a blood flow rate measuring unit 14.

The biosignal sensing device 10 may further include other biosignal measuring means as necessary in addition to the configuration shown in FIG. 2. The configuration related to the measurement of biosignals is a general known technique, and a detailed description thereof will be omitted.

The electrocardiogram measuring unit 12 measures an electrocardiogram of the subject and transmits the electrocardiogram to the controller 40. The term “electrocardiogram” represents a record of electrical changes that occur locally due to cardiac activity. The electrocardiogram is typically measured by inducing an electric potential by means of electrodes attached to a specific part of the body surface.

The blood flow rate measuring unit 14 is a device for measuring the rate of flow of blood passing through blood vessels in a unit time, and may use a photophethysmography sensor. In addition, the blood flow rate measuring unit 14 may be implemented in various ways, such as an electromagnetic blood flow meter, an ultrasonic blood flow meter, or the like.

The user input device 20 serves as a user interface that receives information related to operation from a user who can use the present system and delivers the information to the controller 40. Herein, the user is not limited to an operator who performs external counterpulsation treatment through the operation of the system, and the user may include a subject who receives the treatment.

The user input device 20 receives an initial maximum compression value that can be applied to the subject by the compression device 30 before performing the external counterpulsation by the system and transmits the initial maximum compression value to the controller 40. The user input device 20 may be implemented in various ways, such as a key button, a touch pad, or the like.

The compression device 30 is a device that wraps around the limbs of the subject under the control of the controller 40 and compresses or decompresses the wrapped limbs. The compression device 30 may be comprised of three compression devices, such as a calf compression device, a lower-thigh compression device, and an upper-thigh compression device. However, if necessary, the compression device 30 may be implemented as one or two compression devices, and the wearing position of the compression device 30 may vary.

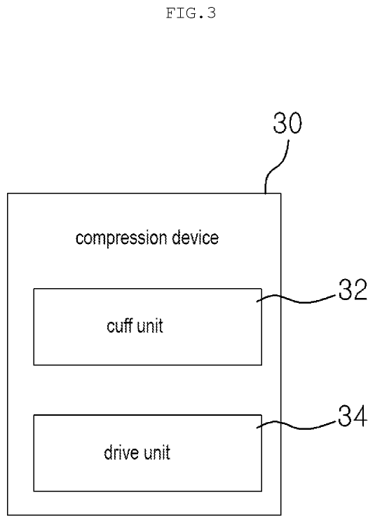

Hereinafter, the compression device 30 will be described in detail with reference to FIG. 3.

As shown in FIG. 3, the compression device 30 may include a cuff unit 32 wrapping around the limbs, and a drive unit 34 driving the cuff unit 32. The drive unit 34 drives the cuff unit 32 under the control of the control unit 40 to allow the cuff unit to compress or decompress the limbs.

The drive unit 34 may be implemented by an electric drive method, such as an electric motor, a solenoid, or the like. However, if necessary, the drive unit 34 may be implemented by a pneumatic drive method.

When the compression device for compressing and decompressing the limbs is implemented by an electric drive method, there is an advantage of reducing noise and vibration over a conventional hydraulic drive method.

The controller 40 controls the overall operation of the present system. The controller is configured to sense biosignals such as an electrocardiogram signal and a blood flow signal through the biosignal sensing device 10, and perform external counterpulsation in response to the sensed biosignals.

The controller 40 configures compression intensity on the basis of an initial maximum compression value configured before performing the external counterpulsation and starts the external counterpulsation on the basis of the configured compression intensity. Herein, the initial maximum compression value may be set by the operator or the like via the user input device 20.

Herein, the initial maximum compression value may be configured by applying test compression to the limbs for a predetermined test period with predetermined test intensity before performing the external counterpulsation.

The controller controls the compression device 30 to apply the test compression to the limbs for the predetermined test period with the predetermined test intensity and configures the initial maximum compression value specified to the subject through the test compression.

The controller 40 configures compression intensity on the basis of the initial maximum compression value configured as above and controls the compression device 30 to start the external counterpulsation on the basis of the configured compression intensity.

The controller 40 calculates compression timing and decompression timing for the external counterpulsation by analyzing the electrocardiogram signal and the blood flow rate signal among the biosignals sensed by the biosignal sensing device 10, and controls the compression device 30 on the basis of the calculated compression timing and decompression timing.

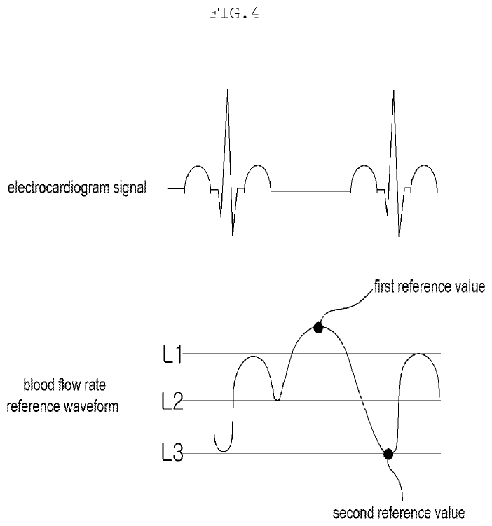

FIG. 4 is a view showing a blood flow rate reference waveform calculated on the basis of an analyzed electrocardiogram signal of the subject. As shown in FIG. 4, the controller 40 calculates a blood flow rate reference waveform for a blood flow rate during late diastole and systole, the blood flow rate being achievable by performing the external counterpulsation, using a result of analyzing the electrocardiogram signal and the blood flow rate signal.

The blood flow rate reference waveform is a waveform of blood flow rate according to an electrocardiogram signal in which the effect of the external counterpulsation is maximized. The blood flow rate reference waveform may vary depending on the subject. As an example, the blood flow rate reference waveform may be determined differently depending on the subject such that the aortic pressure is increased to a predetermined level at a predetermined time of diastole and the aortic pressure is reduced to a predetermined level at a predetermined time of systole.

In the blood flow rate reference waveform of FIG. 4, a peak value of the blood flow rate signal during late diastole is configured as a first reference value, and a valley value of the blood flow rate signal during systole is configured as a second reference value.

The controller 40 controls compression timing T1 and decompression timing T2 such that the waveform of the sensed blood flow rate signal matches the blood flow rate reference waveform, and controls the compression device 30 on the basis of the controlled compression timing T1 and decompression timing T2.

In detail, the controller 40 controls the compression timing T1 and the decompression timing T2 such that the value during late diastole in the waveform of the blood flow rate signal sensed by the biosignal sensing device 10 matches the first reference value of the blood flow rate reference waveform of FIG. 4 and the value during systole in the waveform of the blood flow rate signal sensed by the biosignal sensing device 10 matches the second reference value.

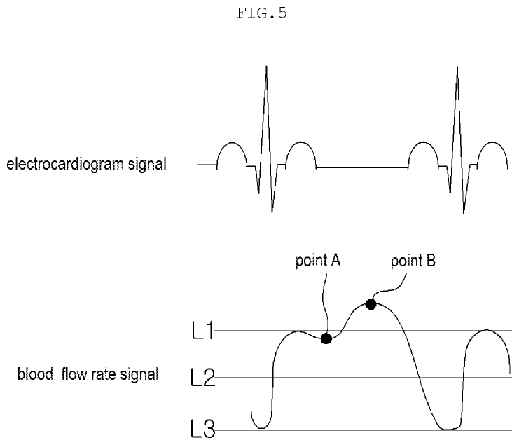

Hereinafter, the control of the compression timing T1 and the decompression timing T2 will be described in detail with reference to FIGS. 5 to 8.

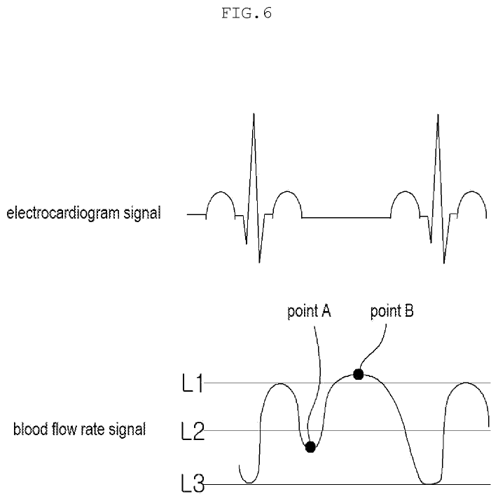

The control of the compression timing T1 will be described with reference to FIGS. 5 and 6.

As shown in FIG. 5, the controller 40 divides the amplitude of the blood flow rate signal into a level L1, a level L2, and a level L3 in order from the highest to the lowest, and when a value (point A) of the blood flow rate signal at the beginning of late diastole is between the level L1 and the level L2 and a value (point B) of the blood flow rate signal at the end of late diastole is near the first reference value of FIG. 4, controls the compression timing by retarding the compression timing T1.

As shown in FIG. 6, the controller 40 controls the compression timing T1 by advancing the compression timing when the value (point A) of the blood flow rate signal at the beginning of late diastole is between the level L2 and the level L3 and the value (point B) of the blood flow rate signal at the end of late diastole is less than the first reference value of FIG. 4.

As described above, the external counterpulsation system 1 according to the present embodiment can automatically control the compression timing and the decompression timing, thus solving the problem of inconvenience that the operator has to control the compression and decompression timings while continuously monitoring procedure as in the related art, and enabling operators without specialized knowledge to use the system easily.

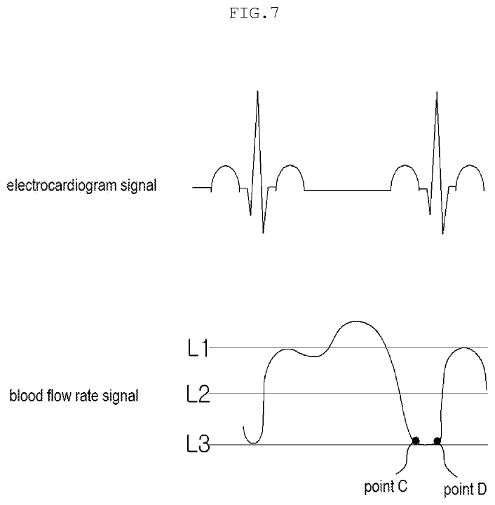

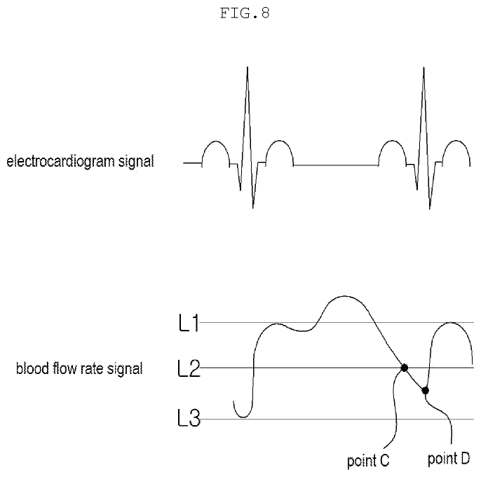

The control of the decompression timing T2 will be described with reference to FIGS. 7 and 8.

As shown in FIG. 7, the controller 40 controls the decompression timing T2 by retarding the decompression timing when a value (point C) of the blood flow rate signal at the beginning of systole and a value (point D) of the blood flow rate signal at the end of systole are near the level L3.

As shown in FIG. 8, the controller 40 controls the decompression timing T2 by advancing the decompression timing when the value (point C) of the blood flow rate signal at the beginning of systole is near the level L2 and the value (point D) of the blood flow rate signal at the end of systole is between the level L2 and the level L3.

As described above, the external counterpulsation system 1 according to the present embodiment can automatically control the compression timing and the decompression timing, thus solving the problem of inconvenience that the operator has to control the compression and decompression timings while continuously monitoring procedure as in the related art, and enabling operators without specialized knowledge to use the system easily.

The controller 40 configures compression intensity on the basis of a preconfigured initial maximum compression value and starts performing external counterpulsation on the basis of the configured compression intensity.

Herein, the initial maximum compression value may be configured by applying test compression to the limbs for a predetermined test period with predetermined test intensity before performing the external counterpulsation.

The controller 40 controls the compression device 30 to apply the test compression to the limbs for the predetermined test period with the predetermined test intensity before performing the external counterpulsation and configures the initial maximum compression value specified to the subject through the test compression.

As such, the controller 40 can automatically configure the initial maximum compression value through the test compression, thus ensuring that, unlike the related art technology, the presence of the operator is not necessarily required and an error that may occur when initial compression intensity is manually input is avoided.

Furthermore, the controller 40 can configure the initial compression intensity at maximum level to be personalized to the subject, thus ensuring that enhanced therapeutic effect of the external counterpulsation is provided.

The controller 40 performs the test compression while incrementally increasing the test intensity for the predetermined test period, and configures the initial maximum compression value on the basis of the increased test intensity when compression due to the increased test intensity is determined to exceed an acceptable compression range which is an acceptable range to the subject. As an example, test intensity before the increased test intensity may be configured as the initial maximum compression value.

As an example, the controller 40 may configure an initial value of test intensity within a range of 70 to 90 mmHg and configure a maximum limit value of the test intensity within a range of 300 to 350 mmHg, to incrementally increase the test intensity by a value within a range of 15 to 25 mmHg. Herein, the initial value of test intensity may be configured as 80 mmHg, the maximum limit value of the test intensity may be configured as 300 mmHg, and the amount of incremental increase of the test intensity may be configured as 20 mmHg.

In addition, the controller 40 may control the compression device 30 to perform test compression for 0.5 seconds for a test period of about 1 second and to perform release of the test compression for about 0.5 seconds.

The controller 40 determines that compression due to the increased test intensity exceeds the acceptable compression range which is the acceptable range to the subject when a notification signal associated with pain in the subject from the user input device 20 is received in the process of performing the test compression while incrementally increasing the test intensity for the predetermined test period. In this case, the controller 40 may configure the initial maximum compression value on the basis of the increased test intensity.

In other words, the controller 40 may determine that compression due to the increased test intensity exceeds the acceptable compression range when the notification signal associated with pain in the subject from a user intention input unit 22 is received, and may configure the initial maximum compression value on the basis of the increased test intensity.

On the other hand, the controller 40 controls the value of initially configured compression intensity by analyzing the electrocardiogram signal and the blood flow rate signal among the biosignals sensed by the biosignal sensing device 10 and controls the compression device 30 to perform the external counterpulsation on the basis of the controlled compression intensity.

In detail, the controller 40 calculates the peak value of the blood flow rate signal during late diastole of the subject using a result of analyzing the electrocardiogram signal and the blood flow rate signal, controls the compression intensity by decreasing the initially configured compression intensity when the calculated peak value of the blood flow rate signal is greater than a predetermined value, and controls the compression intensity by increasing the compression intensity with the initial maximum compression value as an upper limit when the calculated peak value of the blood flow rate signal is less than the predetermined value.

As described above, the external counterpulsation system 1 according to the present embodiment can automatically control the compression intensity applied to the limbs during a procedure. This ensures that, unlike the related art technology, the problem of pain in the subject caused by inability to automatically control the compression intensity during a procedure is solved, and that enhanced therapeutic effect is provided.

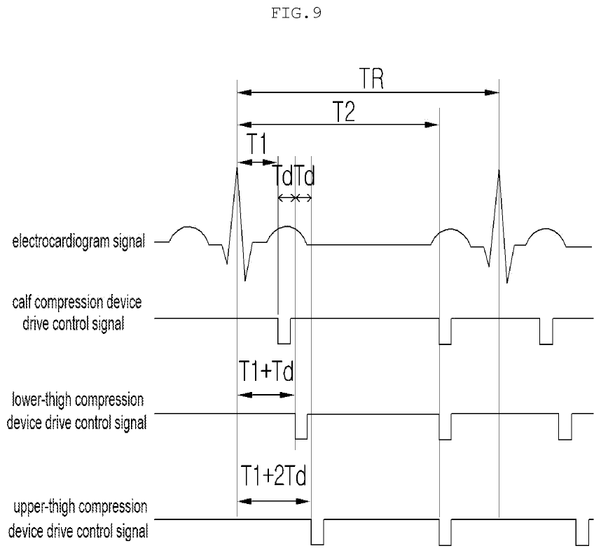

Hereinafter, the operations of compression and decompression by the controller 40 will be described with reference to FIG. 9.

An operation in which the controller 40 controls the compression device 30 on the basis of the compression timing T1 and the decompression timing T2 controlled above. Herein, the compression device 30 will be described by taking an example comprised three compression devices, such as a calf compression device, a lower-thigh compression device, and an upper-thigh compression device.

FIG. 9 is a view showing an operation in which the controller 40 controls the compression device 30 according to the compression timing T1 and the decompression timing T2. In FIG. 9, there is shown the waveform of drive control signals of the controller 40, the drive control signals being generated in association with the compression timing T1 and the decompression timing T2.

As shown in FIG. 9, the controller 40 performs compression sequentially from the calf compression device, the lower-thigh compression device, and the upper-thigh compression device of the compression device 30, and performs decompression simultaneously.

The controller 40 outputs a drive control signal to the compression device 30 such that the compression by the calf compression device is performed at timing “T1”. Next, the controller outputs a drive control signal to the compression device 30 such that the compression by the lower-thigh compression device is performed at timing “T1+Td”. Subsequently, the controller outputs a drive control signal to the compression device 30 such that the compression by the upper-thigh compression device is performed at timing “T1+2Td”. A drive control signal for decompression is simultaneously output to the three compression devices at timing “T2”.

Herein, timing “Td” may be variously controlled depending on the mechanistic characteristics of the compression device and the positions where the three compression devices are placed on the limbs.

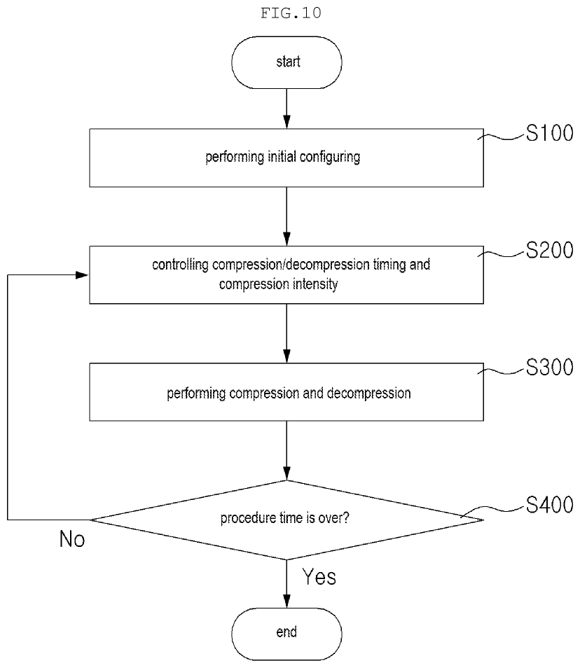

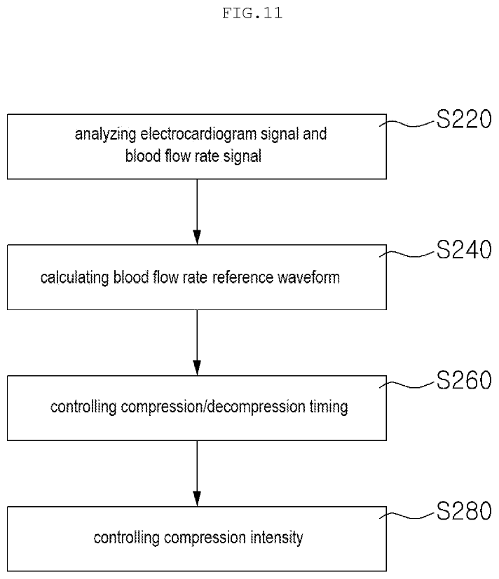

Hereinafter, a method of controlling an external counterpulsation system according to an embodiment of the present invention will be described with reference to FIGS. 10 and 11.

As shown in FIG. 10, a method of controlling an external counterpulsation system includes an initial configuring step (S100), a compress/decompression timing control and compression intensity control step (S200), and a compression/decompression performing step (S300). An external counterpulsation operation is performed in such a manner that the compression/decompression timing control and compression intensity control step (S200) and the compression/decompression performing step (S300) are repeated until an initially configured procedure time is reached (S400). The method of controlling the external counterpulsation system 1 is performed by the controller 40.

First, as in step S100, the external counterpulsation system 1 configures time input via the user input device 20 as a procedure time of the external counterpulsation, and configures compression intensity on the basis of a preconfigured initial maximum compression value. The initial maximum compression value may be determined by applying test compression to limbs for a predetermined test period with predetermined test intensity before performing the external counterpulsation.

Next, as in step S200, the external counterpulsation system 1 analyzes biosignals, such as electrocardiogram and blood flow rate, transmitted from a biosignal sensing device 10 to control compression/decompression timing such that compression and decompression of the limbs for the external counterpulsation are performed, and controls the compression intensity configured on the basis of the initial maximum compression value in step S100.

Hereinafter, step S200 will be described in detail with reference to FIG. 11.

First, the external counterpulsation system 1 analyzes an electrocardiogram signal and a blood flow rate signal among the biosignals sensed by the biosignal sensing device 10 (S220).

Next, the external counterpulsation system 1 calculates a blood flow rate reference waveform for blood flow rate during late diastole and systole, the blood flow rate being achievable by performing the external counterpulsation, using a result of analyzing the electrocardiogram signal and the blood flow rate signal (S240).

The blood flow rate reference waveform is as shown in FIG. 4. In the blood flow rate reference waveform, a peak value of the blood flow rate signal during late diastole is configured as a first reference value, and a valley value of the blood flow rate signal during systole is configured as a second reference value.

Next, the external counterpulsation system 1 controls the compression timing T1 and the decompression timing T2 for the external counterpulsation such that the waveform of the blood flow rate signal sensed by the biosignal sensing device 10 matches the blood flow rate reference waveform calculated in step S240 (S260).

The external counterpulsation system 1 controls the compression timing T1 and the decompression timing T2 such that the value during late diastole in the waveform of the blood flow rate signal sensed by the biosignal sensing device 10 matches the first reference value of the blood flow rate reference waveform of FIG. 4 and the value during systole in the waveform of the blood flow rate signal sensed by the biosignal sensing device 10 matches the second reference value.

The control of the compression timing T1 and the decompression timing T2 is shown in FIGS. 5 to 8 and a detailed description thereof is as described above.

Next, the external counterpulsation system 1 controls the compression intensity configured on the basis of the initial maximum compression value in step S100 by using the result of analyzing the electrocardiogram signal and the blood flow rate signal (S280).

According to step S300, the external counterpulsation system 1 performs the external counterpulsation by controlling the compression device 30 on the basis of the compression timing T1 and the decompression timing T2 and the compression intensity controlled in step S200.

As in step S400, the external counterpulsation system 1 performs steps S200 and S300 until the procedure time of the external counterpulsation configured in step S100.

As described above, the external counterpulsation system and the method of controlling the same according to the present embodiments are characterized by automatically controlling the compression timing and the decompression timing. This ensures that, unlike the related art, the problem of inconvenience that the operator has to control the compression and decompression timings while continuously monitoring procedure is solved, and that operators without specialized knowledge can use the system easily.

Furthermore, the external counterpulsation system and the method of controlling the same are characterized by automatically configuring the initial maximum compression value through the test compression. This ensures that, unlike the related art technology, the presence of the operator is not necessarily required and an error that may occur when the initial compression intensity is manually input is avoided. The present invention is also characterized by configuring the initial compression intensity at maximum level to be personalized to the subject, thus ensuring that enhanced therapeutic effect of the external counterpulsation is provided.

Furthermore, the external counterpulsation system and the method of controlling the same are characterized by automatically controlling the compression intensity applied to the limbs during a procedure. This ensures that, unlike the related art technology, the problem of pain in the subject caused by inability to automatically control the compression intensity during a procedure is solved, and that enhanced therapeutic effect is provided.

### INDUSTRIAL APPLICABILITY

The present invention can find wide application in an external counterpulsation system.

1. An external counterpulsation system that senses biosignals to monitor a heart condition and performs external counterpulsation on the basis of the monitored heart condition, the external counterpulsation system comprising:
a compression device wrapping around limbs of a subject to compress or decompress the wrapped limbs; and
a controller configured to calculate compression timing and decompression timing for the external counterpulsation by analyzing an electrocardiogram signal and a blood flow rate signal among the sensed biosignals, and control the compression device on the basis of the calculated compression timing and decompression timing.

2. The external counterpulsation system of claim 1, wherein the controller calculates a blood flow rate reference waveform for a blood flow rate during late diastole and systole, the blood flow rate being achievable by performing the external counterpulsation, using a result of analyzing the electrocardiogram signal and the blood flow rate signal, controls the compression timing and the decompression timing for the external counterpulsation system such that a waveform of the blood flow rate signal of the sensed biosignals matches the blood flow rate reference waveform, and controls the compression device on the basis of the controlled compression timing and decompression timing.

3. The external counterpulsation system of claim 2, wherein the controller configures a peak value of a blood flow rate signal during late diastole in the blood flow rate reference waveform as a first reference value, configures a valley value of the blood flow rate signal during systole in the blood flow rate reference waveform as a second reference value, and controls the compression timing and the decompression timing such that a value during late diastole in the waveform of the blood flow rate signal of the sensed biosignals matches the first reference value and a value during systole in the waveform of the blood flow rate signal of the sensed biosignals matches the second reference value.

4. The external counterpulsation system of claim 3, wherein the controller divides an amplitude of the blood flow rate signal into a level L1, a level L2, and a level L3 in order from the highest to the lowest, controls the compression timing by retarding the compression timing when a value of the blood flow rate signal at the beginning of late diastole is between the level L1 and the level L2 and a value of the blood flow rate signal at the end of late diastole is near the first reference value, and controls the compression timing by advancing the compression timing when the value of the blood flow rate signal at the beginning of late diastole is between the level L2 and the level L3 and the value of the blood flow rate signal at the end of late diastole is less than the first reference value.

5. The external counterpulsation system of claim 3, wherein the controller controls the decompression timing by retarding the decompression timing when a value of the blood flow rate signal at the beginning of systole and a value of the blood flow rate signal at the end of systole are near the level L3, and controls the decompression timing by advancing the decompression timing when the value of the blood flow rate signal at the beginning of systole is near the level L2 and the value of the blood flow rate signal at the end of systole is between the level L2 and the level L3.

6. The external counterpulsation system of claim 1, wherein the controller configures compression intensity on the basis of a preconfigured initial maximum compression value and starts performing the external counterpulsation on the basis of the configured compression intensity, and the controller controls a value of the configured compression intensity by analyzing the electrocardiogram signal and the blood flow rate signal of the sensed biosignals and controls the compression device to perform the external counterpulsation on the basis of the controlled compression intensity.

7. The external counterpulsation system of claim 6, wherein the controller calculates a peak value of the blood flow rate signal at the end of late diastole of the subject using a result of analyzing the electrocardiogram signal and the blood flow rate signal, controls the compression intensity by decreasing the compression intensity when the calculated peak value of the blood flow rate signal is greater than a predetermined value, and controls the compression intensity by increasing the compression intensity with the initial maximum compression value as an upper limit when the calculated peak value of the blood flow rate signal is less than the predetermined value.

8. The external counterpulsation system of claim 6, wherein the initial maximum compression value is configured by controlling the compression device to apply the test compression to the limbs for the predetermined test period with the predetermined test intensity before performing the external counterpulsation.

9. A method of controlling an external counterpulsation system that senses biosignals to monitor a heart condition and performs external counterpulsation on the basis of the monitored heart condition by controlling a compression device wrapping around limbs of a subject to compress and decompress the wrapped limbs, the method comprising:
analyzing an electrocardiogram signal and a blood flow rate signal among the sensed biosignals;
calculating a blood flow rate reference waveform for a blood flow rate during late diastole and systole, the blood flow rate being achievable by performing the external counterpulsation, using a result of analyzing the electrocardiogram signal and the blood flow rate signal;
controlling compression timing time and decompression timing for the external counterpulsation; and
controlling the compression device on the basis of the controlled compression timing and decompression timing.

10. The method of claim 9, further comprising:
configuring compression intensity on the basis of a preconfigured initial maximum compression value and starting performing the external counterpulsation on the basis of the configured compression intensity; and
controlling the configured compression intensity by using a result of analyzing the electrocardiogram signal and the blood flow rate signal and performing the external counterpulsation on the basis of the controlled compression intensity.
